SEQUENCE LISTING (13 sequences)

SEQ ID NO: 1 - DNA, 26 bp, Artificial Sequence
  lktCAf primer
gcattgaattgatcaactaatacttg

SEQ ID NO: 2 - DNA, 25 bp, Artificial Sequence
  lktCAr primer
caaggtttctagaaagatttttcgg

SEQ ID NO: 3 - DNA, 31 bp, Artificial Sequence
  lktCAAdelf primer
gatcaattgaaagctgttgaagaaattatcg

SEQ ID NO: 4 - DNA, 29 bp, Artificial Sequence
  lktCAAdelr primer
atacaattgattcataatttgcactcgat

SEQ ID NO: 5 - DNA, 48 bp, Artificial Sequence
  lktRBSr primer
caacaattgattcataatttgcctcctataattattctaaattaggtc

SEQ ID NO: 6 - DNA, 1068 bp, Artificial Sequence
  5′ deltalktCA PCR fragment
gcattgaattgatcaactaatacttggtttttcaagtgagttgcaatgcctaaaccatcaccaaaatagtttggattattgattttctcccctacaaaatctagcccttcgtgttttcttgccatctcagccaataccggcacatcgccaaaaatagcatcaattcgcccattttgcacatctaaaatagcattttgataagaggcataagatttcacattgtactcttttttctcttttgctaaatagtgttggtaagtagtcccattttgcacaccaatcgttttcaccttagcaaaatctgtatcttttttcgcaatgaaggcagcagagcttggaaagtaaggctcgctaaataatacttgtttcttacgtggttccgtaatacccatacctgaaattgcagcatcaaattgtttttgttttaggctttggattaagctatcaaaaggttggctatggaatgtacaatttgcattcatctctttacagatagcatttgcaatatccacatcaaaaccgataatttctcccttctcttcggtcatttcaaatggaggatagcttggctccatcacaaatttgatatcttgtgcctgcgcagtaaccacacacccgaataaaagggtcaaaagtgtttttttcataaaaagtccctgtgttttcattataaggattaccactttaacgcagttactttcttaaaaaaagtcttcttttcataaagtttgttttatgtcatacaaacacatcaaattgagatgtagtttctcaatcctcttgattcctctatctcaaaaaaacaacccaaaagaaaaaagaaaagtatatgttacattaatattacaatgtaattattttgtttaatttccctacattttgtataactttaaaacactcctttttctcttctgattatataaaagacaaaaaatacaatttaagctacaaaaaacaacaaaaaacaacaaaaaacacgacaataagatcgagtaatgattatattatgttataatttttgacctaatttagaataattatcgagtccaaattatgaatcaattgtat

SEQ ID NO: 7 - DNA, 1298 bp, Artificial Sequence
  3′ deltalktCA PCR fragment
gatcaattgaaagctgttgaagaaattatcggtacatcacataacgatatctttaaaggtagtaagttcaatgatgcctttaacggtggtgatggtgtcgatactattgacggtaacgacggcaatgaccgcttatttggtggtaaaggcgatgatattctcgatggtggaaatggtgatgattttatcgatggcggtaaaggcaacgacctattacacggtggcaagggcgatgatattttcgttcaccgtaaaggcgatggtaatgatattattaccgattctgacggcaatgataaattatcattctctgattcgaacttaaaagatttaacatttgaaaaagttaaacataatcttgtcatcacgaatagcaaaaaagagaaagtgaccattcaaaactggttccgagaggctgattttgctaaagaagtgcctaattataaagcaactaaagatgagaaaatcgaagaaatcatcggtcaaaatggcgagcggatcacctcaaagcaagttgatgatcttatcgcaaaaggtaacggcaaaattacccaagatgagctatcaaaagttgttgataactatgaattgctcaaacatagcaaaaatgtgacaaacagcttagataagttaatctcatctgtaagtgcatttacctcgtctaatgattcgagaaatgtattagtggctccaacttcaatgttggatcaaagtttatcttctcttcaatttgctagagcagcttaatttttaatgattggcaactctatattgtttcacacattatagagttgccgttttattttataaaaggagacaatatggaagctaaccatcaaaggaatgatcttggtttagttgccctcactatgttggcacaataccataatatttcgcttaatccggaagaaataaaacataaatttgatcttgacggaaaagggctttctttaactgcttggcttttagctgcaaaatcgttagcgttgaaagcgaaacacattaaaaaagagatttcccgcttacacttggtgaatttaccggcattagtttggcaagataacggtaaacattttttattggtaaaagtggataccgataataaccgctatttaacttacaatttggaacaagatgctccacaaattctgtcacaagacgaatttgaagcctgctatcaagggcagttaattttggtcacgtccagagcttccgtagtaggtcaattagcaaagttcgatttcacctggtttattccggcggtgatcaaataccgaaaaatctttctagaaaccttg

SEQ ID NO: 8 - DNA, 2354 bp, Artificial Sequence
  Complete deltaLKTCA with original RBS
gcattgaattgatcaactaatacttggtttttcaagtgagttgcaatgcctaaaccatcaccaaaatagtttggattattgattttctcccctacaaaatctagcccttcgtgttttcttgccatctcagccaataccggcacatcgccaaaaatagcatcaattcgcccattttgcacatctaaaatagcattttgataagaggcataagatttcacattgtactcttttttctcttttgctaaatagtgttggtaagtagtcccattttgcacaccaatcgttttcaccttagcaaaatctgtatcttttttcgcaatgaaggcagcagagcttggaaagtaaggctcgctaaataatacttgtttcttacgtggttccgtaatacccatacctgaaattgcagcatcaaattgtttttgttttaggctttggattaagctatcaaaaggttggctatggaatgtacaatttgcattcatctctttacagatagcatttgcaatatccacatcaaaaccgataatttctcccttctcttcggtcatttcaaatggaggatagcttggctccatcacaaatttgatatcttgtgcctgcgcagtaaccacacacccgaataaaagggtcaaaagtgtttttttcataaaaagtccctgtgttttcattataaggattaccactttaacgcagttactttcttaaaaaaagtcttcttttcataaagtttgttttatgtcatacaaacacatcaaattgagatgtagtttctcaatcctcttgattcctctatctcaaaaaaacaacccaaaagaaaaaagaaaagtatatgttacattaatattacaatgtaattattttgtttaatttccctacattttgtataactttaaaacactcctttttctcttctgattatataaaagacaaaaaatacaatttaagctacaaaaaacaacaaaaaacaacaaaaaacacgacaataagatcgagtaatgattatattatgttataatttttgacctaatttagaataattatcgagtccaaattatgaatcaattgaaagctgttgaagaaattatcggtacatcacataacgatatctttaaaggtagtaagttcaatgatgcctttaacggtggtgatggtgtcgatactattgacggtaacgacggcaatgaccgcttatttggtggtaaaggcgatgatattctcgatggtggaaatggtgatgattttatcgatggcggtaaaggcaacgacctattacacggtggcaagggcgatgatattttcgttcaccgtaaaggcgatggtaatgatattattaccgattctgacggcaatgataaattatcattctctgattcgaacttaaaagatttaacatttgaaaaagttaaacataatcttgtcatcacgaatagcaaaaaagagaaagtgaccattcaaaactggttccgagaggctgattttgctaaagaagtgcctaattataaagcaactaaagatgagaaaatcgaagaaatcatcggtcaaaatggcgagcggatcacctcaaagcaagttgatgatcttatcgcaaaaggtaacggcaaaattacccaagatgagctatcaaaagttgttgataactatgaattgctcaaacatagcaaaaatgtgacaaacagcttagataagttaatctcatctgtaagtgcatttacctcgtctaatgattcgagaaatgtattagtggctccaacttcaatgttggatcaaagtttatcttctcttcaatttgctagagcagcttaatttttaatgattggcaactctatattgtttcacacattatagagttgccgttttattttataaaaggagacaatatggaagctaaccatcaaaggaatgatcttggtttagttgccctcactatgttggcacaataccataatatttcgcttaatccggaagaaataaaacataaatttgatcttgacggaaaagggctttctttaactgcttggcttttagctgcaaaatcgttagcgttgaaagcgaaacacattaaaaaagagatttcccgcttacacttggtgaatttaccggcattagtttggcaagataacggtaaacattttttattggtaaaagtggataccgataataaccgctatttaacttacaatttggaacaagatgctccacaaattctgtcacaagacgaatttgaagcctgctatcaagggcagttaattttggtcacgtccagagcttccgtagtaggtcaattagcaaagttcgatttcacctggtttattccggcggtgatcaaataccgaaaaatctttctagaaaccttg

SEQ ID NO: 9 - DNA, 2354 bp, Artificial Sequence
  Complete deltalktCA (with consensus RBS)
gcattgaattgatcaactaatacttggtttttcaagtgagttgcaatgcctaaaccatcaccaaaatagtttggattattgattttctcccctacaaaatctagcccttcgtgttttcttgccatctcagccaataccggcacatcgccaaaaatagcatcaattcgcccattttgcacatctaaaatagcattttgataagaggcataagatttcacattgtactcttttttctcttttgctaaatagtgttggtaagtagtcccattttgcacaccaatcgttttcaccttagcaaaatctgtatcttttttcgcaatgaaggcagcagagcttggaaagtaaggctcgctaaataatacttgtttcttacgtggttccgtaatacccatacctgaaattgcagcatcaaattgtttttgttttaggctttggattaagctatcaaaaggttggctatggaatgtacaatttgcattcatctctttacagatagcatttgcaatatccacatcaaaaccgataatttctcccttctcttcggtcatttcaaatggaggatagcttggctccatcacaaatttgatatcttgtgcctgcgcagtaaccacacacccgaataaaagggtcaaaagtgtttttttcataaaaagtccctgtgttttcattataaggattaccactttaacgcagttactttcttaaaaaaagtcttcttttcataaagtttgttttatgtcatacaaacacatcaaattgagatgtagtttctcaatcctcttgattcctctatctcaaaaaaacaacccaaaagaaaaaagaaaagtatatgttacattaatattacaatgtaattattttgtttaatttccctacattttgtataactttaaaacactcctttttctcttctgattatataaaagacaaaaaatacaatttaagctacaaaaaacaacaaaaaacaacaaaaaacacgacaataagatcgagtaatgattatattatgttataatttttgacctaatttagaataattataggaggcaaattatgaatcaattgaaagctgttgaagaaattatcggtacatcacataacgatatctttaaaggtagtaagttcaatgatgcctttaacggtggtgatggtgtcgatactattgacggtaacgacggcaatgaccgcttatttggtggtaaaggcgatgatattctcgatggtggaaatggtgatgattttatcgatggcggtaaaggcaacgacctattacacggtggcaagggcgatgatattttcgttcaccgtaaaggcgatggtaatgatattattaccgattctgacggcaatgataaattatcattctctgattcgaacttaaaagatttaacatttgaaaaagttaaacataatcttgtcatcacgaatagcaaaaaagagaaagtgaccattcaaaactggttccgagaggctgattttgctaaagaagtgcctaattataaagcaactaaagatgagaaaatcgaagaaatcatcggtcaaaatggcgagcggatcacctcaaagcaagttgatgatcttatcgcaaaaggtaacggcaaaattacccaagatgagctatcaaaagttgttgataactatgaattgctcaaacatagcaaaaatgtgacaaacagcttagataagttaatctcatctgtaagtgcatttacctcgtctaatgattcgagaaatgtattagtggctccaacttcaatgttggatcaaagtttatcttctcttcaatttgctagagcagcttaatttttaatgattggcaactctatattgtttcacacattatagagttgccgttttattttataaaaggagacaatatggaagctaaccatcaaaggaatgatcttggtttagttgccctcactatgttggcacaataccataatatttcgcttaatccggaagaaataaaacataaatttgatcttgacggaaaagggctttctttaactgcttggcttttagctgcaaaatcgttagcgttgaaagcgaaacacattaaaaaagagatttcccgcttacacttggtgaatttaccggcattagtttggcaagataacggtaaacattttttattggtaaaagtggataccgataataaccgctatttaacttacaatttggaacaagatgctccacaaattctgtcacaagacgaatttgaagcctgctatcaagggcagttaattttggtcacgtccagagcttccgtagtaggtcaattagcaaagttcgatttcacctggtttattccggcggtgatcaaataccgaaaaatctttctagaaaccttg

SEQ ID NO: 10 - DNA, 21 bp, Artificial Sequence
  ts ori forward primer
gatccctttttctgtaatctg

SEQ ID NO: 11 - DNA, 21 bp, Artificial Sequence
  ts ori reverse primer
gatcataggctcaattctcgc

SEQ ID NO: 12 - DNA, 18 bp, Artificial Sequence
  kanamycin resistance forward primer
atgagccatattcaacgg

SEQ ID NO: 13 - DNA, 22 bp, Artificial Sequence
  kanamycin resistance reverse primer
tcagaaaaactcatcgagcatc

(19) United States
(12) Patent Application Publication
(10) Pub. No.: US 2017/0022510 A1
(43) Pub. Date: Jan. 26, 2017

(54) Attenuated Mannheimia haemolytica

(71) Applicant: Biotechnology Research and Development Corporation, Peoria, IL (US)

(72) Inventors: Robert E. Briggs, Boone, IA (US); Fred M. Tatum, Ames, IA (US)

(21) Appl. No.: 15/076,140

(22) Filed: Mar. 21, 2016

Related U.S. Application Data
(63) Continuation-in-part of application No. 14/934,737, filed on Nov. 6, 2015.
Application No. 14/934,737 is a continuation of application No. 14/075,169, filed on Nov. 8, 2013, now Pat. No. 9,370,561.
(60) Provisional application No. 61/723,979, filed on Nov. 8, 2012.

(51) Int. Cl. C12N 15/74; C07K 14/285; C12N 9/80

(52) U.S. Cl. CPC C12N 15/74; C12N 9/80; C07K 14/285; C12Y 305/01

(57) ABSTRACT

This disclosure provides attenuated M. haemolytica strains useful for providing immunity against M. haemolytica.

This application is a continuation-in-part of Ser. No. 14/934,737 filed on Nov. 6, 2015, which is a continuation of Ser. No. 14/075,169 filed on Nov. 8, 2013, which claims priority to Ser. No. 61/723,979 filed on Nov. 8, 2012.

This application incorporates by reference the contents of an 11.2 kb text file created on Mar. 21, 2016 and named “sequencelisting.txt,” which is the sequence listing for this application.

### TECHNICAL FIELD

This disclosure relates generally to attenuated bacteria and their use in vaccines.

### BRIEF DESCRIPTION OF THE DRAWINGS

FIG. 1 depicts the stepwise construction of M. haemolytica serotypes 1 and 6 in-frame leukotoxin deletion mutants.

FIG. 2A agarose gel electrophoresis of PCR products from M. haemolytica LktCABD operon showing truncated LktCA (lane 2) and wildtype LktCA (lane 3).

FIG. 2B Western blot analysis of truncated LktA expressed by M. haemolytica D153ΔlktCA4-707, vaccine strain. Lane 1, marker; lane 2, 5 μl of culture supernatant containing truncated LktA (*=27 kDa, M. haemolytica D153ΔlktCA4-707); lane 3, 5 μl of culture supernatant containing wildtype LktA (*=102 kDa, M. haemolytica D153 parent strain). Mutants without the modified rbs express poorly and are not shown.

### DETAILED DESCRIPTION

This disclosure provides attenuated M. haemolytica strains which can be used to prepare vaccine compositions useful for protection against M. haemolytica. In some embodiments, the M. haemolytica is serotype A1 (D153). In other embodiments, the M. haemolytica is serotype A6 (D174).

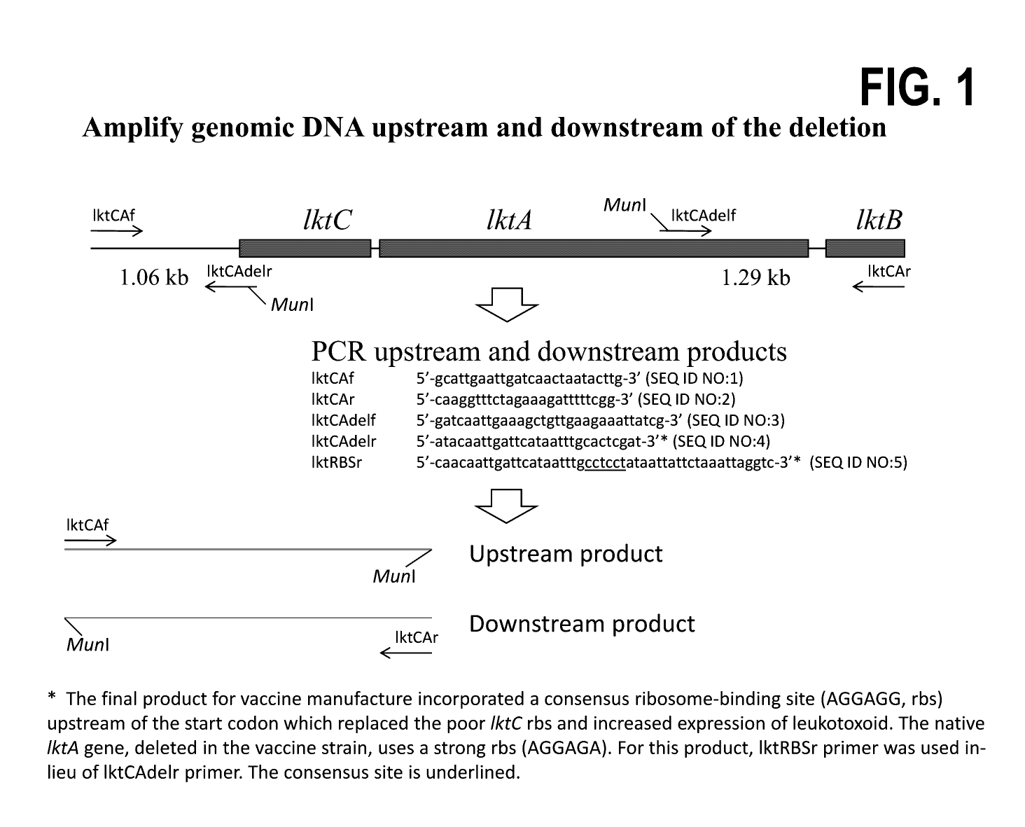

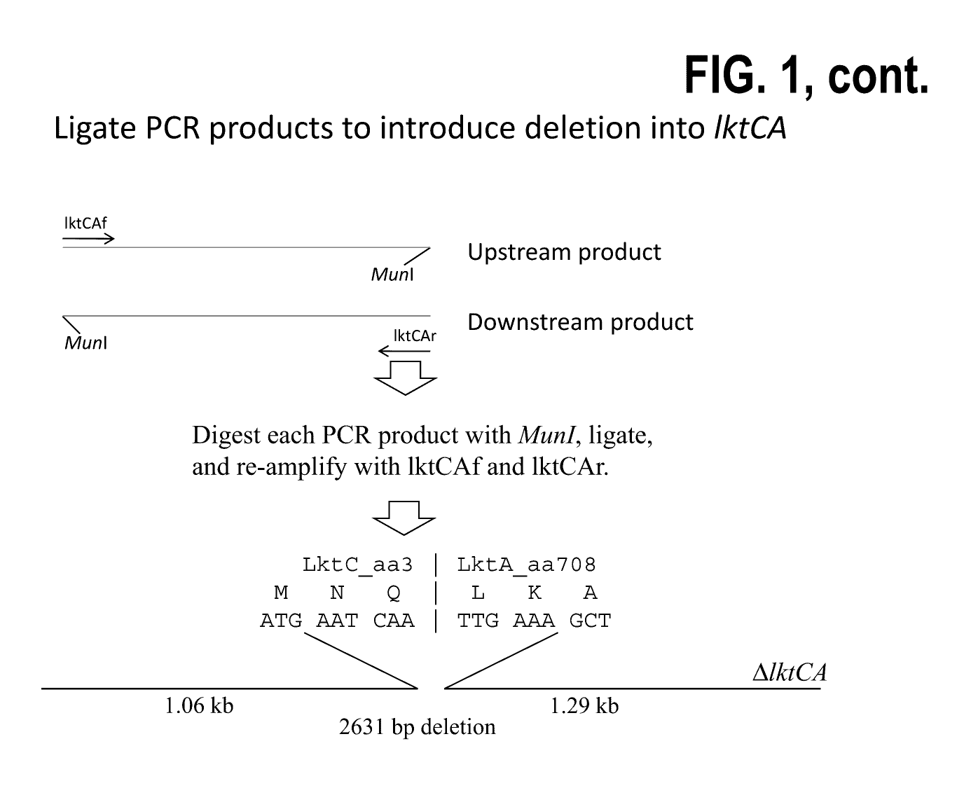

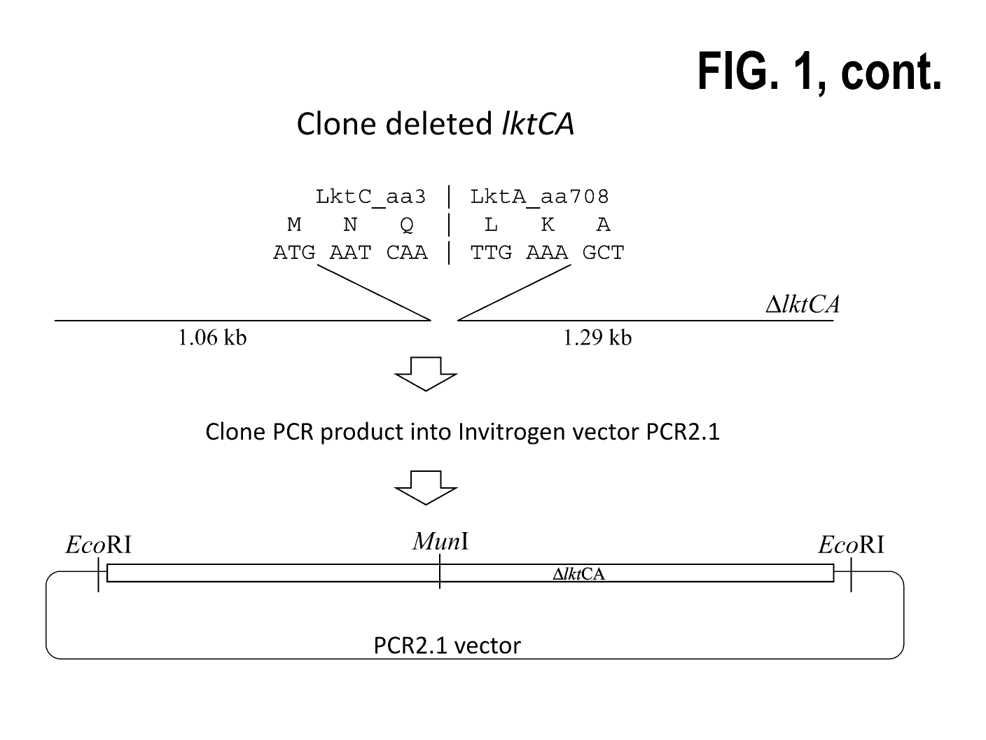

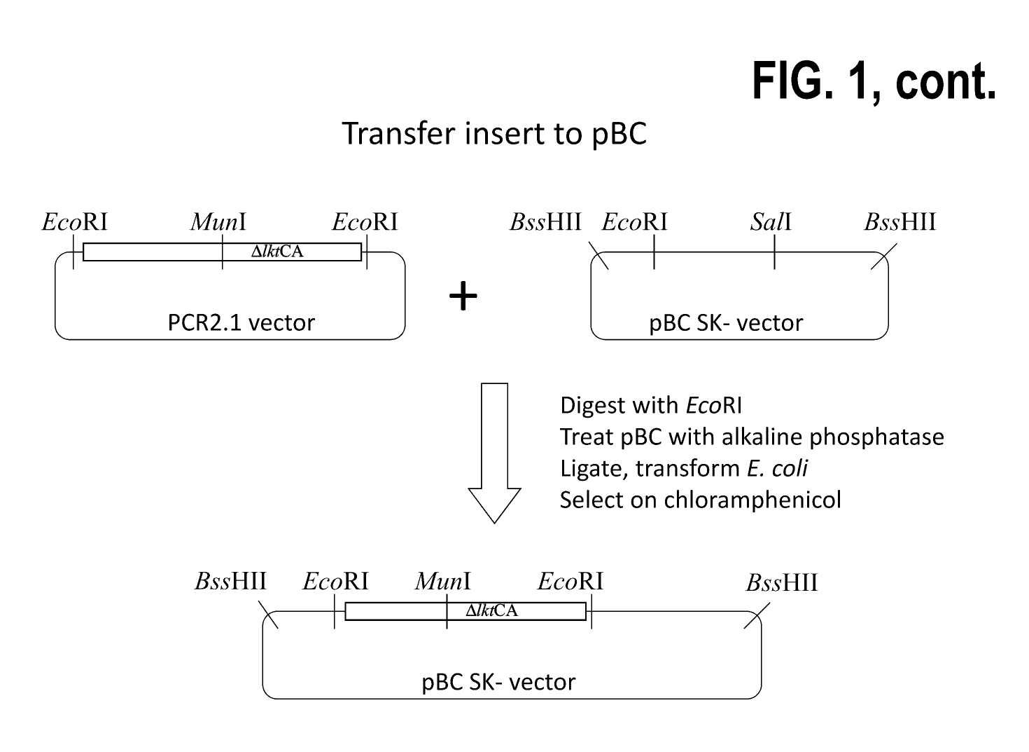

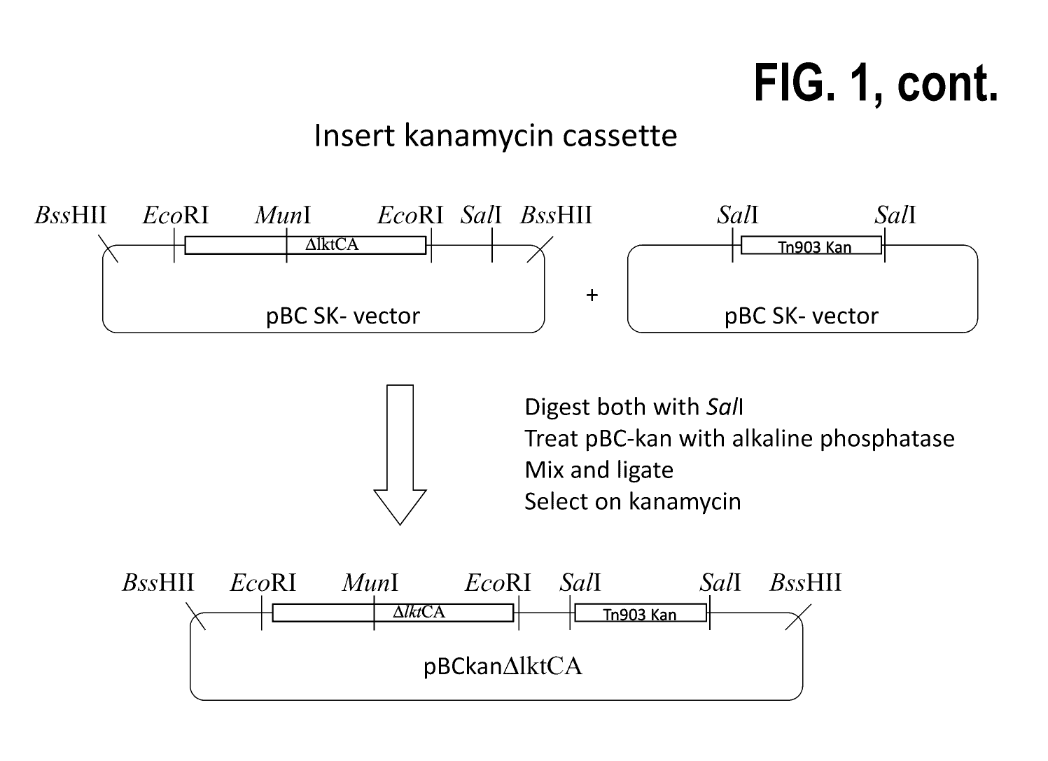

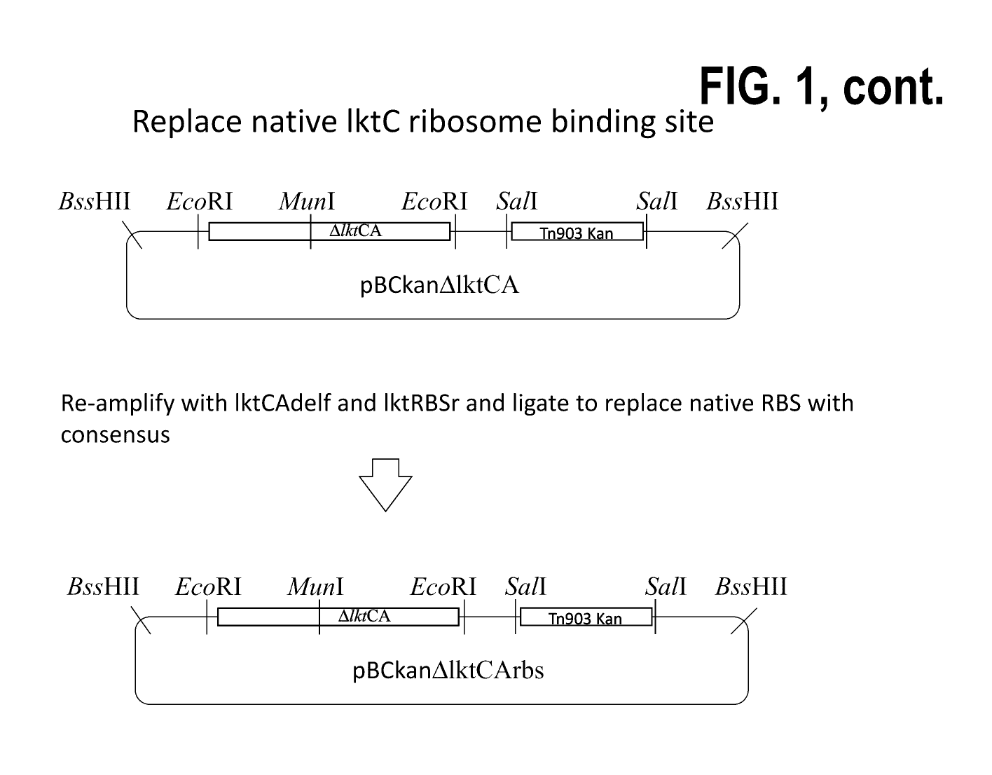

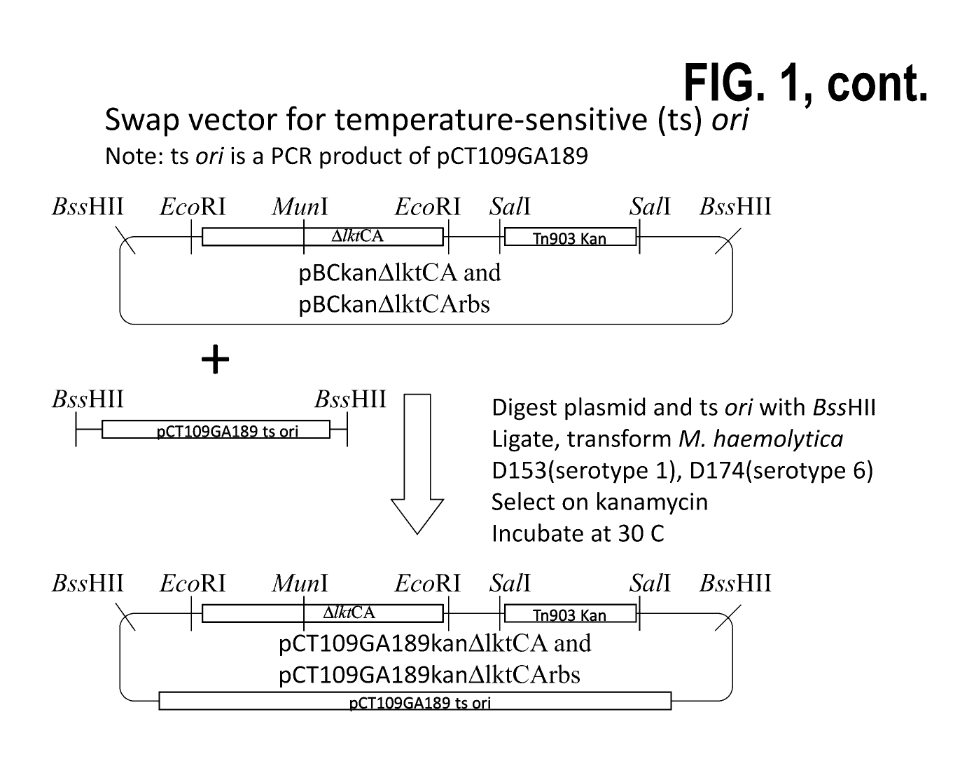

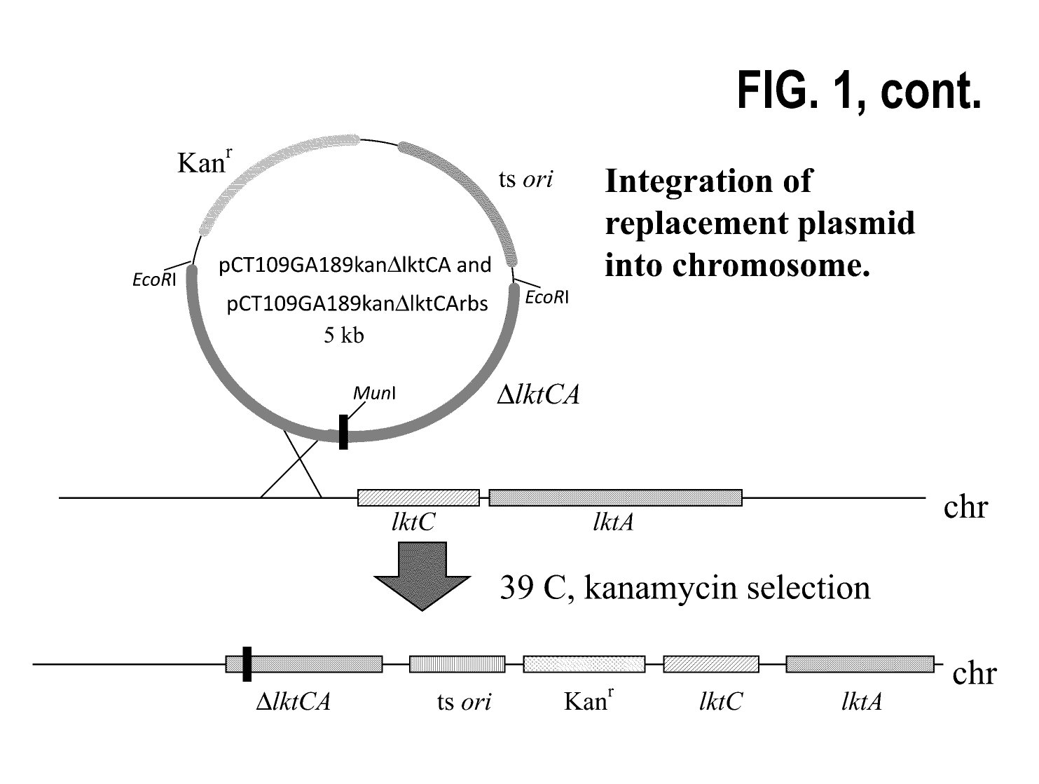

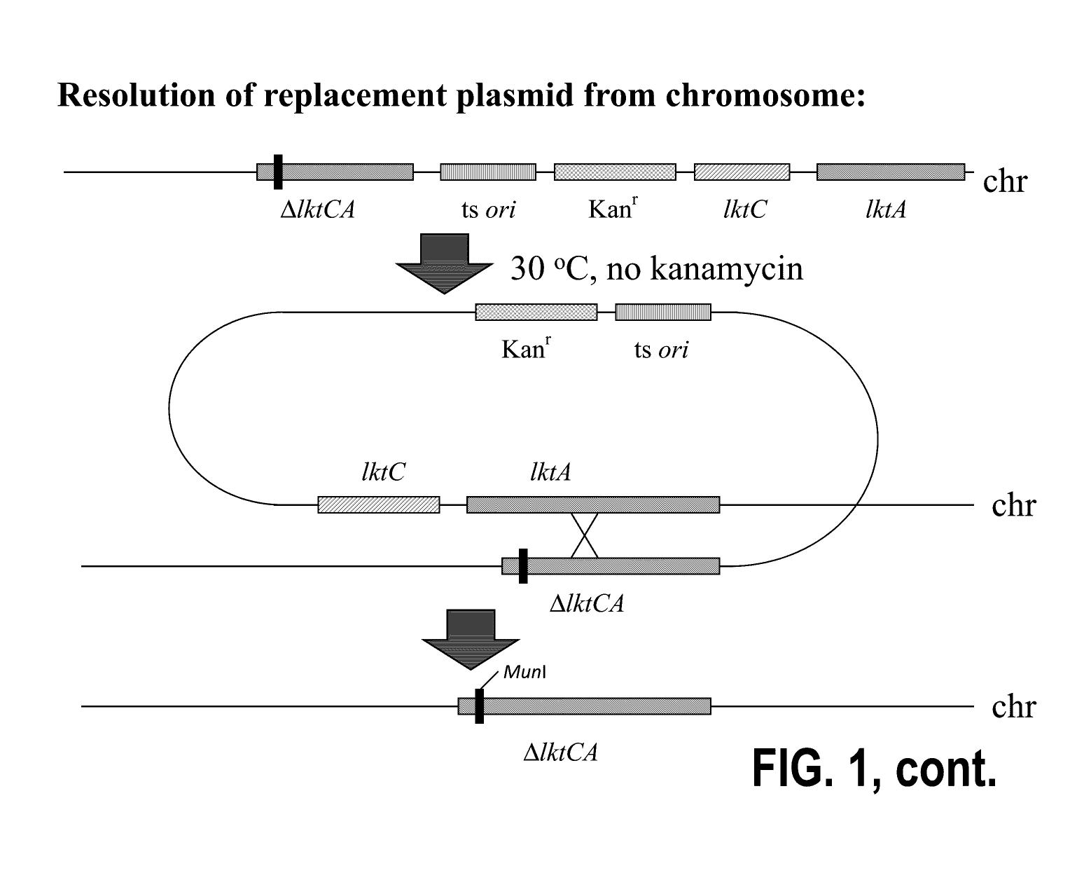

To attenuate the bacterium, we deleted nucleotides within the LktCA locus, which encodes an enzyme acylase (LktC) and leukotoxin A (LktA), the bacterium's principal virulence factor. This deletion can be amplified by polymerase chain reaction (PCR) and the secretion of a truncated LktA can be detected on a Western blot to determine if the bacterium is the mutant or wildtype. The genetic engineering is summarized in FIG. 1. All reagents, including the shuttle vectors pCR2.1, pBC SK, pSK, and pCT109GA189 ts ori, and the E. coli DH11S host cell, are known to and accessible by persons skilled in the art. Studies reported in U.S. 2014/0170190, incorporated herein by reference, confirm the immunogenicity of the mutant strains.

### Construction of lktCA Deletion

pCT109GA189-KanΔlktCA and pCT109GA189-KanΔlktCA-rbs were constructed as outlined in FIG. 1. Briefly, two DNA fragments, upstream (1.06 kb, SEQ ID NO:6) and downstream (1.29 kb, SEQ ID NO:7) were PCR amplified from M. haemolytica strain NADC D153. Whole cells were used as template using the primer sets, lktCAf (SEQ ID NO:1)/lktCAdelr (SEQ ID NO:4) and lktCAr (SEQ ID NO:2)/lktCAdelf (SEQ ID NO:3). The PCR products were phenol-chloroform-extracted to inactivate Taq polymerase and then digested with MunI prior to ligation. The ligation products were PCR amplified with primer pair lktCAf/lktCAr and the products were cloned using a commercially available vector (PCR2.1, Invitrogen, Carlsbad, Calif.) according to the manufacturer's instructions.

A product containing an approximately 2.3 kb insert was selected and proper sequence across the deletion was confirmed by DNA sequencing and designated pTAΔlktCA. The 2.3 kb deleted leukotoxin insert in pTAΔlktCA was transferred into pBC by digestion with EcoRI and ligation into the unique EcoRI site to form pBCΔlktCA. A kanamycin cassette derived from pUC4K was placed into the EcoR1 site of pBC SK-(Stratagene Inc.) to generate pBCKan. The kanamycin cassette of pBCKan was transferred into pBCΔlktCA by digestion with SalI and ligation into the unique SalI site of pBCΔlktCA to form pBCKanΔlktCA. This product was amplified by PCR using primer pair lktCAdelf (SEQ ID NO:3) and lktRBSr (SEQ ID NO:5) to replace the native lktC ribosome binding site (RBS) with a consensus RBS. The PCR product was phenol-chloroform-extracted to inactivate Taq polymerase and then digested with MunI prior to ligation onto itself to form pBCKanΔlktCArbs. Proper sequence adjacent to the deletion was confirmed by DNA sequencing. Finally the pBC plasmid backbone of both pBCKanΔlktCA and pBCKanΔlktCArbs was replaced with the temperature-sensitive plasmid origin of replication from pCT109GA189 (Briggs and Tatum, Appl. Environ. Microbiol. 71, 7187-95, 2005, incorporated herein by reference) by ligating BssHII-digested preparations of each to generate pCT109GA189KanΔlktCA and pCT109GA189KanΔlktCArbs.

Because the temperature-sensitive plasmid origin functions poorly in E. coli cloning hosts, these final ligation products were introduced directly into M. haemolytica. Prior cloning steps used E. coli DH11S (Life Technologies, Rockville, Md.) as the cloning host.

Electrocompetent M. haemolytica serotype A1 D153 cells and serotype A6 D174 (parental strains) were transformed with pCT109GA189KanΔlktCA and pCT109GA189KanΔlktCArbs by previously described methods except unmethylated ligation product was directly introduced into the competent cells (Briggs and Tatum, 2005). Briefly, cells were made electrocompetent by growing them to logarithmic phase in 100 ml of Columbia broth (Difco Laboratories, Detroit, Mich.) at 37° C. with gentle shaking. The cells were pelleted by centrifugation at 5,000×g and washed in 100 ml of 272 mM sucrose at 0° C., and the pellet was suspended in an equal volume of 272 mM sucrose at 0° C. After electroporation, cells recovered overnight in 10 ml Columbia broth at 30° C. Growth (50 μl) was spread onto Columbia agar plates containing 50 μg/ml kanamycin, which were then incubated 36 hours at 30° C.

Individual colonies were passed to broth containing 50 μg/ml kanamycin and incubated overnight at 30° C. Growth (100 μl) was passed again to Columbia agar plates with kanamycin which were incubated overnight at 39° C. Individual colonies were passed to trypticase soy agar (TSA) plates containing 5% defibrinated sheep blood (BA plates, incubated overnight at 39° C.) and to Columbia broth without selection (incubated overnight at 30° C.).

Growth in broth was streaked for isolation on BA plates and passed again in broth at 30° C. Non-hemolytic colonies which were kanamycin-sensitive were detected on BA plates after 1 to 3 passages without selection. Representative colonies from each recipient strain and replacement plasmid were selected for further study.

Non-hemolytic mutants were grown in Columbia broth at 37° C. for 3 hours and harvested in late logarithmic growth. Supernatants were dotted onto nitrocellulose along with supernatants from the wild-type parent and a leukotoxin-negative isogenic mutant. After appropriate blocking and washing, the blot was probed with monoclonal anti-leukotoxin antibody 2C9-1E8 (neutralizing antibody produced by NADC, Ames, Iowa). Mutant products containing the native ribosome binding site were found to express low levels of protein reactive to monoclonal antibody, less than that produced by the wild-type parent strain. Products which contained the new ribosome binding site produced much higher levels of reactive protein.

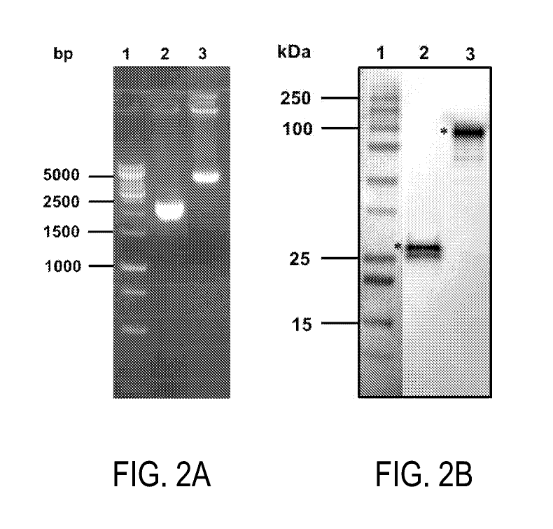

Supernatants of two products expressing high levels of leukotoxin were concentrated 15-fold on a 10,000 MW filter (Centriprep, Amicon). The concentrates (1.5 μl) were subjected to SDS-PAGE, blotted to nitrocellulose, and probed with antibody 2C9-1E8. Western blot analysis indicated a new protein reactive with neutralizing anti-leukotoxin monoclonal antibody at an apparent molecular weight consistent with the 27 kDa predicted protein (truncated LktA) product. The mutant M. haemolytica serotype A1 was designated as D153ΔlktCA4-707, the mutant M. haemolytica serotype A6 was designated as D174ΔlktCA4-707, which refers to the amino acid positions in LktC and LktA respectively where the deleted region begins and ends. Gene deletion was characterized by PCR amplification using LktCAf (SEQ ID NO:1) and LktCAr (SEQ ID NO:2) primers, which flank the deletion site. As indicated by the gel image, PCR amplification yielded the expected approximately 2.3 kb for truncated LktCA, and approximately 5.0 kb for the wild-type bacterium (FIG. 2A).

Finally, these representative mutants, and single-crossover controls, were analyzed by PCR to demonstrate the absence of temperature-sensitive origin and kanamycin-resistance cassette. PCR was performed with primers within ts ori (forward primer 5′-GATCCCTTTTTCTGTAATCTG-3′, SEQ ID NO:10; reverse primer 5′-GATCATAGGCTCAATTCTCGC-3′, SEQ ID NO:11) and kanamycin resistance (forward primer 5′-ATGAGCCATATTCAACGG-3′, SEQ ID NO:12; reverse primer 5′-TCAGAAAAACTCATCGAGCATC-3′, SEQ ID NO:13) genes confirmed those elements were no longer present in the final LktCA mutant for Master Seed (MS). Five microliters of the concentrated culture supernatant was run on a SDS-PAGE system, blotted onto PVDF membrane and probed using mouse anti-LktA, neutralizing antibody 2C9-1E8 (1:1000) as primary antibody. Goat anti-mouse IgG (1:4000) coupled with alkaline phosphatase was used as secondary antibody and developed in a substrate solution containing NBT/BCIP for 1-5 min (FIG. 2B).

1. A deletion mutant M. haemolytica bacterium comprising a deletion with the LktCA gene locus encoding acylase (LktC) and leukotoxin A (LktA).

2. The deletion mutant M. haemolytica bacterium of claim 1, which is an M. haemolytica serotype A1 bacterium.

3. The deletion mutant M. haemolytica bacterium of claim 2, which secretes a truncated form of the LktA protein having an amino acid sequence encoded by the nucleic acid sequence set forth in SEQ ID NO:8 or SEQ ID NO:9.

4. The deletion mutant M. haemolytica bacterium of claim 1, which is an M. haemolytica serotype A6 bacterium.

5. The deletion mutant M. haemolytica bacterium of claim 4, which secretes a truncated form of the LktA protein having an amino acid sequence encoded by the nucleic acid sequence set forth in SEQ ID NO:8 or SEQ ID NO:9.
